(19) United States
(12) Patent Application Publication
(10) Pub. No.: US 2017/0356893 A1
(43) Pub. Date: Dec. 14, 2017

(54) MOISTURE DETECTION SENSOR, DEFECT DETECTION SENSOR, AND SENSOR ARRAY USING SAME

(71) Applicant: KOREA INSTITUTE OF CERAMIC ENGINEERING AND TECHNOLOGY, Jinju-si (KR)

(72) Inventors: Eun Hae KOO, Daejeon (KR); Hyun Chul KIM, Gimpo-si (KR)

(73) Assignee: KOREA INSTITUTE OF CERAMIC ENGINEERING AND TECHNOLOGY, Jinju-si (KR)

(21) Appl. No.: 15/540,744

(22) PCT Filed: Sep. 11, 2015
(86) PCT No.: PCT/KR2015/009557

§ 371 Date: Jun. 29, 2017

(30) Foreign Application Priority Data
Dec. 30, 2014 (KR) 10-2014-0193643

(51) Int. Cl. G01N 31/22; G01N 21/64; G01N 21/78; G01N 33/00; G01N 21/77; G01N 21/75

(52) U.S. Cl. CPC G01N 31/222; G01N 21/78; G01N 33/00; G01N 21/643; G01N 2021/758; G01N 2033/0078; G01N 2021/7786

(57) ABSTRACT

Provided are a moisture detection sensor, a defect detection sensor, and a sensor array using the same that make use of moisture-sensitive compounds reversibly reacting to water (moisture) to emit fluorescence, thereby reversibly sensing the moisture within a short period of time and also providing a high degree of sensitivity even to an extremely small quantity of moisture. The moisture detection sensor includes one or more moisture-sensitive compounds selected from the group consisting of Calcein, Calcein-AM (Calcein acetoxymethyl ester), and Calcein blue.

### BACKGROUND OF THE INVENTION

### Field of the Invention

The present invention relates to a moisture detection sensor, a defect detection sensor, and a sensor array using the same, and more particularly, to a moisture detection sensor, a defect detection sensor, and a sensor array using the same that make use of moisture-sensitive compounds reversibly reacting to water (moisture) to emit fluorescence, thereby reversibly sensing the moisture within a short period of time and also providing a high degree of sensitivity even to an extremely small quantity of moisture.

### Background of the Related Art

Recently, an organic light-emitting diode OLED, which has been importantly emerged in the field of displays, is widely applied to a variety of products from small cell phones to 55 inch TVs. One of important technologies in the OLED display is a gas barrier technology (water and oxygen blocking or encapsulation technology) related to the life span and durability of the OLED. That is, the OLED is very sensitive to moisture and has an allowable value of 10⁻⁶ g/m² day (the quantity of moisture permeated per 1 square meter of a substrate for a day) in a water vapor transmission rate, WVTR. Currently, the OLED makes use of a glass substrate, so that there is no problem in the water vapor transmission rate of the substrate itself, but water vapor transmission rate problems are emphasizedly solved through the improvements of the barrier characteristics of packaging and sealing materials.

On the other hand, devices such as flexible displays or electronic paper are lightweight, bent or folded, unlike existing hard electronic products, so that it is expected that they will play an important role in the future market. However, such flexible electronic products make use of a plastic (polymer) substrate, thereby causing big problems. That is, the plastic substrate has a structure having a free volume in which a degree of denseness among molecules is low, so that a large quantity of moisture may enter the device through the substrate itself, and in this case, a water vapor transmission rate is more than 10¹ g/m² day. This value is 10⁷ times bigger than the allowable value of the water vapor transmission rate required in the OLED display. Accordingly, new technologies in which a variety of barrier films are disposed on top of the plastic substrate have been developed to prevent the allowable value of the water vapor transmission rate from being exceeded, and representatively, a polymer/ceramic multilayer structure has been suggested.

In addition to the technology for preventing the allowable value of the water vapor transmission rate from being exceeded, on the other hand, a technology for measuring water vapor transmission rate properties of for a developed material is very important. There are four representative technologies for measuring the water vapor transmission rate properties, that is, a transmission rate measurement method, an infrared IR measurement method, mass spectrometry, and a calcium test.

Particularly, the calcium test is a representative method for measuring an extremely low water vapor transmission rate of 10⁻⁴ g/m² day, and this technology measures the water vapor transmission rate using UV-visible light indicating a degree of transparency of opaque calcium through the reaction to moisture. Generally, water vapor is saturated in an inert gas or dry air and is then transferred to a given quantity of reaction material (for example, calcium), so that the degree of transparency of the reaction material (calciumhydro oxide), that is, the water vapor transmission rate is measured and obtained, thereby completing the measurement of the water vapor transmission rate. However, the calcium test obtains the water vapor transmission rate for a small portion corresponding to several centimeters of a test sample, which is not absolute rate, but a relative comparison value, so that it is hard to be applied to the measurement of the water vapor transmission rate of the substrate and the barrier film of the display device produced with a large area.

The IR measurement method is applied under the principle when the energy levels of rotational, vibrational, and translational motions of water molecules correspond to IR wavelengths and light of IR wavelengths is irradiated, the irradiated light is absorbed, and in this case, the IR measurement method is widely used scientifically. Due to the sensitivity limit of the detector, however, it is hard to measure the water vapor transmission rate of 10⁻⁴ g/m² day and under.

The mass spectrometry is capable of measuring the water vapor transmission rate on the basis of a scientific principle, but due to various problems, like the IR measurement method, it is industrially hard to measure the water vapor transmission rate of 10⁻⁴ g/m² day and under. Further, factors giving the biggest influence on the water vapor transmission rate are defects produced on a film. Accordingly, real-time monitoring for the defects is very important to solve the problems related to the water vapor transmission rate.

Accordingly, a demand for a method for measuring a water vapor transmission rate of 10⁻⁴ g/m² day and under within a short period of time is greatly increased around display markets, there are needs to develop a material appearing dramatically big change in optical characteristics even with respect to an extremely small quantity of moisture, to develop a moisture measurement system, and to develop a defect measurement method.

### SUMMARY OF THE INVENTION

Accordingly, the present invention has been made in view of the above-mentioned problems occurring in the prior art, and it is an object of the present invention to provide a moisture detection sensor that reversibly senses moisture within a short period of time, while providing a high degree of sensitivity even to an extremely small quantity of moisture.

It is another object of the present invention to provide a defect detection sensor that is provided with one or more moisture-sensitive compounds comprising a moisture detection sensor serving as a sensing layer.

It is yet another object of the present invention to provide a sensor array that includes a moisture detection sensor or defect detection sensor.

To accomplish the above-mentioned objects, according to a first aspect of the present invention, there is provided a moisture detection sensor including one or more moisture-sensitive compounds selected from the group consisting of Calcein, Calcein-AM (Calcein acetoxymethyl ester), and Calcein blue.

According to the present invention, preferably, the Calcein as the moisture-sensitive compounds is represented by the following chemical formula:

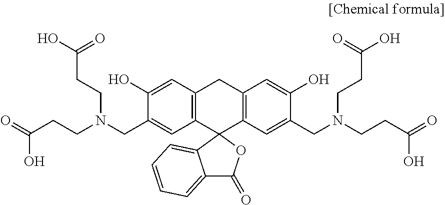 [SMILES: O=C(O)CCN(CCC(=O)O)Cc1cc2c(cc1O)Cc1cc(O)c(CN(CCC(=O)O)CCC(=O)O)cc1C21OC(=O)c2ccccc21]

According to the present invention, the moisture-sensitive compounds reversibly react to moisture.

According to the present invention, the moisture-sensitive compounds emit fluorescence upon the detection of moisture.

According to the present invention, the moisture detection sensor further includes one or more metal selected from the group consisting of Au, Ag, Cu, Co, Rh, Ti and Fe or non-metal elements.

To accomplish the above-mentioned objects, according to a second aspect of the present invention, there is provided a defect detection sensor including a sensing layer having one or more moisture-sensitive compounds selected from the group consisting of Calcein, Calcein-AM (Calcein acetoxymethyl ester), and Calcein blue, wherein the sensing layer emits fluorescence when reacts to moisture.

To accomplish the above-mentioned objects, according to a third aspect of the present invention, there is provided a sensor array for detecting water or defect comprising: a sensor part selected from the moisture detection sensor or the defect detection sensor; a light emitting part for emitting fluorescence from the moisture-sensitive compounds, and a light receiving part for receiving the fluorescence emitted from the moisture-sensitive compounds.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a graph showing fluorescence increments observed with changing moisture contents added dropwise to a DMF solution containing Calcein;

FIG. 2 is a schematic view showing a defect detection sensor according to the present invention and its mode of use thereof; and

FIG. 3 is a photograph of defects of a film laminated on the defect detection sensor to which a moisture-sensitive compounds according to the present invention is applied as a sensing layer.

### DETAILED DESCRIPTION OF THE PREFERRED EMBODIMENTS

Now, an explanation on a moisture detection sensor, a defect detection sensor, and a sensor array using the same according to the present invention will be given with reference to the attached drawings.

A moisture detection sensor according to the present invention includes one or more moisture-sensitive compounds selected from the group consisting of Calcein, Calcein-AM (Calcein acetoxymethyl ester) and Calcein blue.

The compounds are represented by the following first to third chemical formulas:

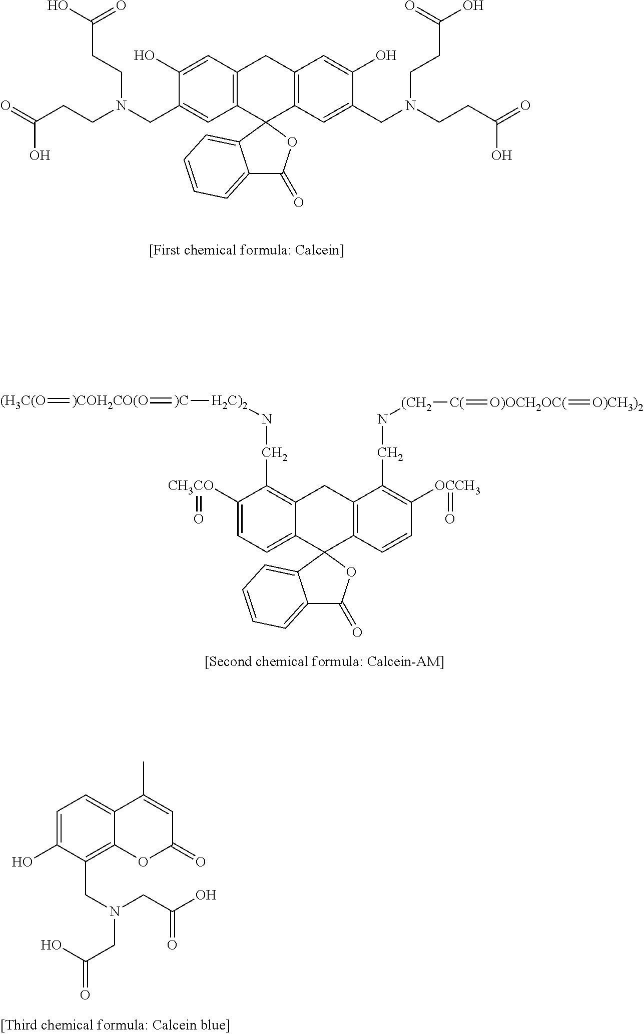

These compounds are known compounds and are commercially available.

The compounds are non-fluorescence substances, but they are converted into substances that emit fluorescence through the addition of water thereto. The fluorescence caused by the addition of water and fluorescence quenching are reversible.

The actions of the compounds according to the present invention will be explained, for example, through the Calcein compound represented by the first chemical formula.

The Calcein is well known as a substance emitting fluorescence. The excitation wavelength of the Calcein compound is 492 nm, and the fluorescence wavelength thereof is 547 nm. When water molecules are absent, the fluorescence of the Calcein is quenched and lost due to an influence of unpaired electrons. However, the Calcein compound is changeable to the following reaction equation through the addition or removal of moisture, and the addition and removal reactions of the moisture are reversible.

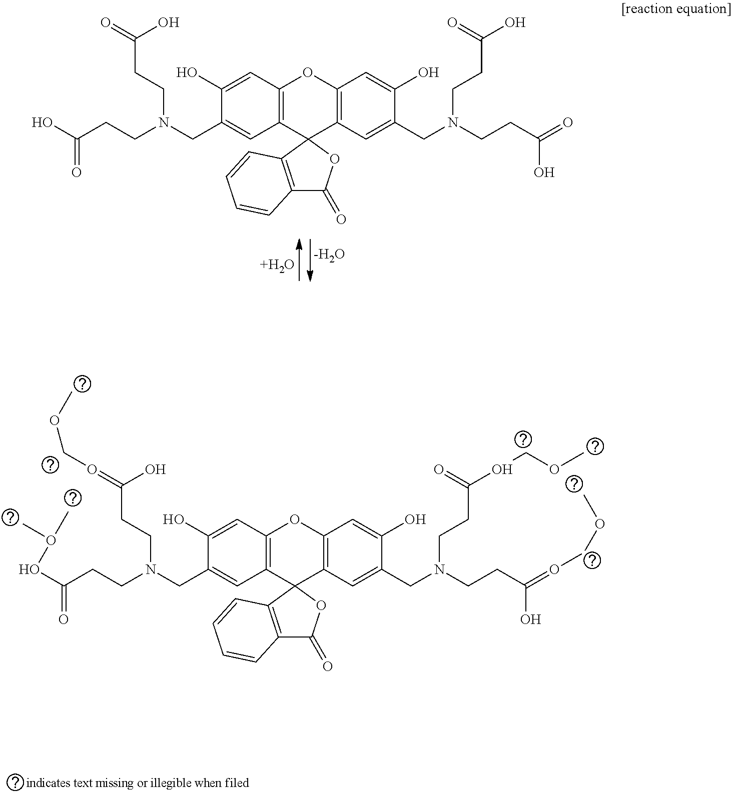

In more detail, the Calcein represented by the first chemical formula has an influence of unpaired electrons of nitrogen atom in the state where water molecules do not exist, so that the fluorescence causes quenching effect, that is, a PET (photo-induced electron transfer) phenomenon, resulting in nonradiative relaxation of energy absorbed in the path that does not exhibit fluorescence. However, in the presence of water molecules, it is believed that the unpaired electrons of the nitrogen atom no longer cause the PET phenomenon, so that the fluorescence of the first chemical formula is turned on. According to the repeated experiments of the inventors, it is found that the moisture detecting sensor using the above compounds is capable of detecting a trace amount of moisture below a water concentration of 100 ng/cc.

On the other hand, the PET phenomenon caused from the moisture-sensitive compounds in the moisture detection sensor according to the present invention can be amplified by bonding to metal or transition metals. At this time, the bond between the moisture-sensitive compounds and the metal or transition metals is a coordination bond forming a complex form, and in this case, examples of the metal or transition metals used are Au, Ag, Cu, Co, Rh, Ti and Fe.

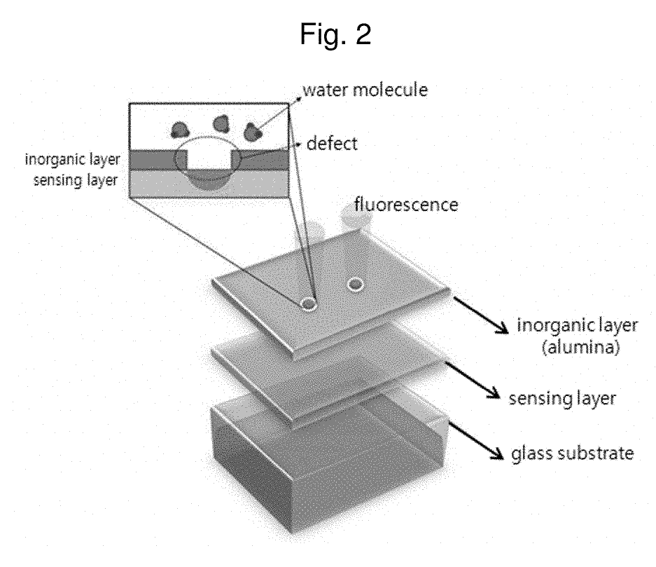

Further, a defect detection sensor according to the present invention is provided with a sensing layer containing the above-mentioned moisture-sensitive compounds. FIG. 2 is a schematic view showing a defect detection sensor according to the present invention and the use state thereof. As shown in FIG. 2, the defect detection sensor includes the sensing layer disposed on a glass substrate, and the sensing layer contains the above-mentioned moisture-sensitive compounds. On the other hand, a film having defects to be detected is laminated on top of the sensing layer, and moisture, which is permeated into the defects on the film, reacts to the moisture-sensitive compounds to cause the emission of fluorescence, so that the defects of the film laminated on top of the sensing layer can be detected. At this time, the positions and sizes of the defects are detected.

According to a preferred embodiment of the present invention, the defect detection sensor may be configured wherein the sensing layer is formed by coating the moisture-sensitive compounds onto a substrate. As the substrate used for this purpose, glass, polyolefin, polyester film or the like can be used. At this time, a coating method for forming the sensing layer is selected appropriately from wet coating like spin coating, bar coating, knife coating, micro gravure coating, and roll coating, thermal vacuum deposition, and sputtering.

According to another preferred embodiment of the present invention, the defect detection sensor may be configured wherein the sensing layer can be used with a hydrophilic polymer that does not participate in the fluorescence mechanisms of the moisture-sensitive compounds. Examples of the hydrophilic polymer that can be used for this purpose include polyethylene oxide (PEO), polyacrylic acid (PAA), and the like. In this case, the sensing layer is formed by preparing a coating solution by dissolving or dispersing the moisture-sensitive compounds and the hydrophilic polymer in an appropriate solvent, and then applying the coating liquid onto a substrate and drying. The above-described hydrophilic polymer can contribute to a uniform dispersion and uniform coating of the water-sensitive compound, for example, without blocking the opportunity for the water-sensitive compound to react with moisture owing to its hydrophilicity.

The defects detected by the defect detection sensor according to the present invention are moisture transmission defects. For example, it may be a moisture transmission cracks, pin holes, scratches, or portions that are thinly formed to a thickness not more than prescribed thickness in a manufacturing process, such as an optical film containing a polarizer film applied to an OLED display device, barrier film and the like.

The defects may penetrate through the film or may be applied to a defect detection sensor of the present invention if the defect has a moisture transmission rate of 10⁻⁶ g/m² day and above, even if the defects does not pass through the film. On the other hand, the defect detection sensor according to the present invention can detect not only the defect passing the moisture in a liquid state but also the defect passing the moisture in a vapor state.

On the other hand, the film where the defects detectable by the defect detection sensor according to the present invention is present includes, for example, an inorganic film formed of silica, silicon, ITO, ZTO, ZnS, GaP, Ta₂O₃, TiO₂, GeO₂, and VOx and an organic film formed of plastic materials such as polyolefin like polyethylene PE or polypropylene PP, polyester like polyethylene terephthalate PET or Polyethylene-naphthalate PEN, polystyrene PS, polyurethane PU, epoxy, polyethersulfone PES, polyimide PI, polyetheretherketone PEEK, polysulfone PSF, polyethylenimine PEI and so on. For example, the film is used as a layer of a part constituting a solar cell, OLED, or semiconductor device, or as a part of a crystal liquid display, flexible display, or flat panel display device.

According to preferred embodiment of the present invention, the defect detection sensor may be composed of only a substrate and a sensing layer formed thereon. In this case, the defect detection sensor is bonded to a film to be detected such as a plastic film, and then, the edges of the bonded body are sealed by known means. After that, the sealed bonded body comes into contact with moisture or air in which moisture is contained, so that the moisture permeated into the film to be detected and reacting to the moisture-sensitive compounds contained in the sensing layer of the defect detection sensor is detected to the form of fluorescence, thereby checking the positions and sizes of the defects existing on the film.

According to another preferred embodiment of the present invention, the defect detection sensor can be used as an optical element like an OLED and solar cell or as a part of an element like a display panel. In this case, the defect detection sensor includes a substrate and a sensing layer, and the substrate is disposed on the underside of a film to be detected and is coated with the moisture-sensitive compounds. In this case, the defects of the film formed on top of the sensing layer are detected directly in the element itself.

According to the present invention, additionally, there is provided a sensor array for detecting water or defect having the above-mentioned moisture or defect detection sensor. More particularly, the sensor array comprises a sensor part having a moisture or defect detection sensor having moisture-sensitive compounds, a light emitting part for emitting fluorescence from the moisture-sensitive compounds, and a light receiving part for receiving the fluorescence emitted from the moisture-sensitive compounds.

Hereinafter, an explanation on the configuration and effects of the present invention will be in more detail given by way of particular examples.

### Example

(1) Manufacturing Compounds Capable of Easily Detecting Water and Film Defects

A synthesis process of compounds using the first chemical formula was as follows. Calcein, 3-3′-Bis[N, N-di(carboxymethy)-aminomethy]fluroescein, and hydrophilic polymer, poly ethylene oxide PEO were dissolved in dimethyl formamide DMF and agitated at a speed of 500 RPM and a temperature of 80° C. for one hour.

(2) Moisture Sensitivity Evaluation

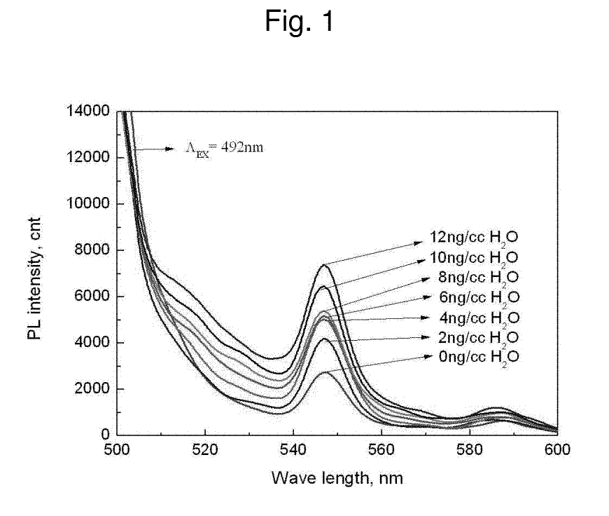

So as to check moisture detection characteristics, fluorescence intensities were measured according to the quantities of moisture through a fluorescence spectrometer (PL spectrometer, model name: S-3100 made by SCINCO). The quantity of the first chemical formula compounds having a concentration of 1×10⁻²[M] and the PEO was adjusted to the amount of 0.05 wt % of DMF. While moisture having a concentration of 20 ppm was being added dropwise, fluorescence increments were observed. The results for the evaluation of moisture sensitivity are provided in FIG. 1.

FIG. 1 is a graph showing fluorescence increments observed while changing the moisture contents in solution containing Calcein at a concentration of 1×10²[M] in DMF solvent. From FIG. 1, if Calcein is exposed to moisture, it emits fluorescence. Further, in addition, it found that the intensity of the fluorescence increases almost linearly with an increase in the moisture contents in which the added moisture contents are within the experimental range, that is, within the range of 2 to 12 ng/cc.

(3) Defect Detection Sensor

So as to check defect characteristics, further, the solution having the compounds of the first chemical formula was spin-coated on the glass, as shown in FIG. 2, and then dried, thereby manufacturing the defect detection sensor. An inorganic alumina layer (having a thickness of 50 nm) was formed on top of the sensing layer of the defect detection sensor through RF magnetron sputtering deposition, and so as to observe defects occurring, after that, the prepared sample was left in the air for one day, so that the moisture in the air could sufficiently react to the compounds of the first chemical formula through the defects on the alumina layer.

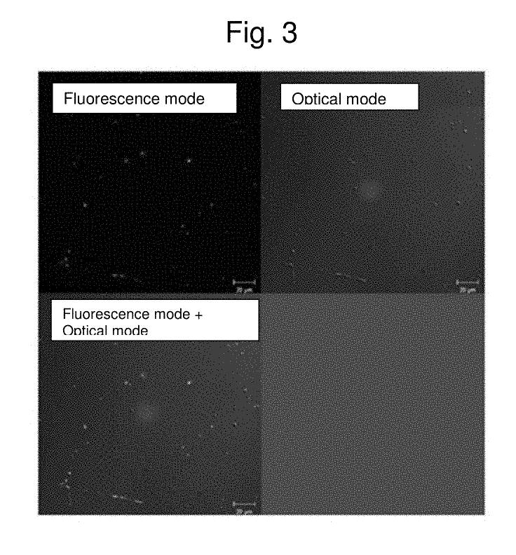

The photograph on which the defects are observed through optical and fluorescence modes using a confocal laser scanning microscope is suggested in FIG. 3. FIG. 3 is a photograph on which defects are observed on the alumina layer formed on the defect detection sensor according to the present invention. It is checked from FIG. 3 that the defects, which are difficult to be observed in the optical mode through the microscope, are clearly recognized in positions and relative sizes when observed in the fluorescence mode.

As described above, the moisture detection sensor and the defect detection sensor according to the present invention are capable of accurately measuring only the quantity of moisture, without having any interference of coexisting gas, thereby providing excellent selectivity, are capable of reversibly reacting to moisture, thereby being continuously reusable to save the cost consumed, and are capable of effectively monitoring the changes in the concentration of moisture.

In addition, the moisture detection sensor and the defect detection sensor according to the present invention are capable of providing excellent sensitivity, precise detection, and rapid response speed. Further, the moisture is easily permeated into the defects, and accordingly, the fluorescence intensities are increased, so that the defects can be easily monitored.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. A moisture detection sensor comprising one or more moisture-sensitive compounds selected from the group consisting of Calcein, Calcein-AM (Calcein acetoxymethyl ester), and Calcein blue.

2. The moisture detection sensor according to claim 1, wherein the Calcein as the moisture-sensitive compounds is represented by the following chemical formula:

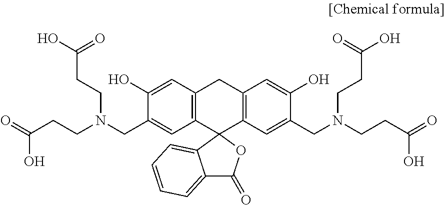 [SMILES: O=C(O)CCN(CCC(=O)O)Cc1cc2c(cc1O)Cc1cc(O)c(CN(CCC(=O)O)CCC(=O)O)cc1C21OC(=O)c2ccccc21]

3. The moisture detection sensor according to claim 1, wherein the moisture-sensitive compounds reversibly react to moisture.

4. The moisture detection sensor according to claim 1, wherein the moisture-sensitive compounds emit fluorescence upon the detection of moisture.

5. The moisture detection sensor according to claim 1, further comprising one or more metal selected from the group consisting of Au, Ag, Cu, Co, Rh, Ti and Fe or non-metal elements.

6. A defect detection sensor comprising a sensing layer having one or more moisture-sensitive compounds selected from the group consisting of Calcein, Calcein-AM (Calcein acetoxymethyl ester), and Calcein blue, wherein the sensing layer emits fluorescence when reacts to moisture.

7. A sensor array for detecting water or defect comprising;
a sensor part including the moisture detection sensor of claim 1,
a light emitting part for emitting fluorescence from the moisture-sensitive compounds, and
a light receiving part for receiving the fluorescence emitted from the moisture-sensitive compounds.

8. A sensor array for detecting water or defect comprising;
a sensor part including the defect detection sensor of claim 6,
a light emitting part for emitting fluorescence from the moisture-sensitive compounds, and
a light receiving part for receiving the fluorescence emitted from the moisture-sensitive compounds.
